(19) United States
(12) Patent Application Publication
(10) Pub. No.: US 2025/0064498 A1
(43) Pub. Date: Feb. 27, 2025

(54) SPLINE-BASED ABLATION CATHETER HAVING RETAINER FEATURE

(71) Applicant: Boston Scientific Scimed, Inc., Maple Grove, MN (US)

(72) Inventors: James V. Kauphusman, Champlin, MN (US); Edward J. Maierhofer, Brooklyn Park, MN (US); Andrew L. DeKock, Ham Lake, MN (US); Nathan P. Hagstrom, Minneapolis, MN (US); Brian Soltis, St. Paul, MN (US)

(21) Appl. No.: 18/810,110

(22) Filed: Aug. 20, 2024

Related U.S. Application Data
(60) Provisional application No. 63/578,529, filed on Aug. 24, 2023.

(51) Int. Cl. A61B 18/00

(52) U.S. Cl. CPC A61B 18/00; A61B 2018/00077; A61B 2018/00083; A61B 2018/00172; A61B 2018/00267; A61B 2018/00351; A61B 2018/00577; A61B 2018/00613; A61B 2018/00839

(57) ABSTRACT

An ablation catheter includes a tubular shaft having a proximal portion and a distal end and an electrode assembly extending from the distal end of the shaft. The electrode assembly includes a plurality of splines each having a spline proximal portion secured to the shaft, and an opposite spline distal portion. Each spline includes an electrically conductive support member partially disposed within an insulating member. The support member has a proximal end portion disposed within the insulating member and a distal end portion extending distally of the insulating member and terminating at a support member distal end. A plurality of sensing electrodes is disposed along each spline. A spline retainer mechanically engages with at least one of the proximal and distal portions of the splines.

### CROSS REFERENCE TO RELATED APPLICATIONS

This application claims priority to U.S. Provisional Patent Application No. 63/578,529 entitled “SPLINE-BASED ABLATION CATHETER HAVING RETAINER FEATURE,” filed Aug. 24, 2023, which is hereby incorporated by reference in its entirety.

### TECHNICAL FIELD

The present disclosure relates to medical systems and methods for ablating tissue in a patient. More specifically, the present disclosure relates to medical systems and methods for ablation of tissue by electroporation.

### BACKGROUND

Ablation procedures are used to treat many different conditions in patients. Ablation can be used to treat cardiac arrhythmias, benign tumors, cancerous tumors, and to control bleeding during surgery. Usually, ablation is accomplished through thermal ablation techniques including radio-frequency (RF) ablation and cryoablation. In RF ablation, a probe is inserted into the patient and radio frequency waves are transmitted through the probe to the surrounding tissue. The radio frequency waves generate heat, which destroys surrounding tissue and cauterizes blood vessels. In cryoablation, a hollow needle or cryoprobe is inserted into the patient and cold, thermally conductive fluid is circulated through the probe to freeze and kill the surrounding tissue. RF ablation and cryoablation techniques indiscriminately kill tissue through cell necrosis, which may damage or kill otherwise healthy tissue, such as tissue in the esophagus, phrenic nerve cells, and tissue in the coronary arteries.

Another ablation technique uses electroporation. In electroporation, or electro-permeabilization, an electrical field is applied to cells in order to increase the permeability of the cell membrane. The electroporation can be reversible or irreversible, depending on the strength of the electric field. If the electroporation is reversible, the increased permeability of the cell membrane can be used to introduce chemicals, drugs, and/or deoxyribonucleic acid (DNA) into the cell, prior to the cell healing and recovering. If the electroporation is irreversible, the affected cells are killed through apoptosis.

Irreversible electroporation can be used as a nonthermal ablation technique. In irreversible electroporation, trains of short, high voltage pulses are used to generate electric fields that are strong enough to kill cells through apoptosis. In ablation of cardiac tissue, irreversible electroporation can be a safe and effective alternative to the indiscriminate killing of thermal ablation techniques, such as RF ablation and cryoablation. Irreversible electroporation can be used to kill targeted tissue, such as myocardium tissue, by using an electric field strength and duration that kills the targeted tissue but does not permanently damage other cells or tissue, such as non-targeted myocardium tissue, red blood cells, vascular smooth muscle tissue, endothelium tissue, and nerve cells. Planning irreversible electroporation ablation procedures can be difficult due to the lack of acute visualization or data indicating which tissues have been irreversibly electroporated, as opposed to reversibly electroporated where tissue recovery can occur over minutes, hours, or days after the ablation is completed.

### SUMMARY

Example 1 is a catheter for ablation of cardiac tissue through irreversible electroporation. The catheter comprises a tubular shaft having a proximal portion and a distal end; an electrode assembly extending from the distal end of the shaft, the electrode assembly including a plurality of splines each having a spline proximal portion secured to the shaft, and an opposite spline distal portion. Each of the splines comprises an electrically conductive support member having a support member proximal portion coupled to the shaft distal end and a support member distal portion terminating at a support member distal end, wherein each of the electrically conductive support members is configured to be operable as a first ablation electrode configured to generate an electric field when a pulsed waveform is delivered to each support member. An electrical insulating member covers the support member proximal portion, the insulating member having an insulating member proximal portion coupled to the shaft distal end; and a plurality of sensing electrodes disposed along the insulating member. A spline retainer mechanically engages with at least one of the support member distal portion or the support member proximal portion of each of the plurality of splines, the spline retainer adapted to mechanically support the splines.

Example 2 is the catheter of Example 1 where the spline retainer includes a distal spline retainer mechanically coupled to the support member distal end of each of the plurality of splines so as to mechanically couple the spline distal portions together.

In Example 3, in the catheter of Example 2, the distal spline retainer is electrically conductive and is electrically coupled to one or more of the support members and configured to form a part of the first ablation electrode.

In Example 4, in the catheter of Example 2, the distal spline retainer is configured to be operable as a distal sensing electrode.

In Example 5, in the catheter of any of Examples 2-4, the distal spline retainer is disposed at a distal tip of the electrode assembly.

In Example 6, in the catheter of any of Examples 2-5, the distal spline retainer is a conductive ball.

In Example 7, in the catheter of any of Examples 5-6, wherein the distal spline retainer includes a first piece proximal to the distal tip mechanically coupled to a second piece distal to the distal tip.

In Example 8, in the catheter of any of claims 1-7, wherein the first ablation electrode is configured as a monopolar ablation electrode.

In Example 9, in the catheter of any of Examples 1-8 further comprising a shaft electrode disposed on the shaft proximate the distal end thereof.

In Example 10, in catheter of Example 9, the first ablation electrode and the shaft electrode are configured to form an electrode pair for bipolar ablation.

In Example 11, in the catheter of any of Examples 1-10, at least one of the spline sensing electrodes is configured to be operable as a second ablation electrode.

In Example 12 catheter of any of claims 1-10, at least one of the spline sensing electrodes is configured to be operable as a second ablation electrode.

In Example 13, in the catheter of any of Examples 1-12, the spline retainer includes a proximal spline retainer mechanically abutted against the support member proximal portions of each of the plurality of splines so as to space apart the spline proximal portions in the expanded and collapsed states.

In Example 14, in the catheter of Example 13, the proximal spline retainer is coupled to and extends from the shaft distal end into an interior space formed by the electrode assembly.

In Example 15, in the catheter of Example 13, the proximal spline retainer is integral to the shaft distal end.

Example 16 is a catheter for ablation of cardiac tissue through irreversible electroporation, the catheter comprising: a tubular shaft having a proximal portion and a distal end; an electrode assembly extending from the distal end of the shaft, the electrode assembly including a plurality of splines each having a spline proximal portion secured to the shaft, and an opposite spline distal portion, each of the splines comprising an electrically conductive support member having a support member proximal portion coupled to the shaft distal end and a support member distal portion terminating at a support member distal end, wherein each of the electrically conductive support members is configured to be operable as a first ablation electrode configured to generate an electric field when a pulsed waveform is delivered to each support member; and an electrical insulating member covering the support member proximal portion, the insulating member having an insulating member proximal portion coupled to the shaft distal end; and a plurality of sensing electrodes disposed along the insulating member; and a spline retainer mechanically engaged with at least one of the support member distal portion or the support member proximal portion of each of the plurality of splines, the spline retainer adapted to mechanically support the splines.

In Example 17, the catheter of Example 16, wherein the spline retainer includes a distal spline retainer mechanically coupled to the support member distal end of each of the plurality of splines so as to mechanically couple the spline distal portions together.

In Example 18, in the catheter of Example 17, the distal spline retainer is electrically conductive and is electrically coupled to one or more of the support members and configured to form a part of the first ablation electrode.

In Example 19, in the catheter of Example 17, the distal spline retainer is configured to be operable as a distal sensing electrode.

In Example 20, the catheter of Example 19, wherein the distal spline retainer is disposed at a distal tip of the electrode assembly.

In Example 21, the catheter of Example 20, wherein the distal spline retainer is a conductive ball.

In Example 22, in the catheter of Example 20, the distal spline retainer includes a first piece proximal to the distal tip mechanically coupled to a second piece distal to the distal tip.

In Example 23, in the catheter of Example 16, the first ablation electrode is configured as a monopolar ablation.

In Example 24, the catheter of Example 16, further comprises a shaft electrode disposed on the shaft proximate the distal end thereof.

In Example 25, in the catheter of Example 24, the first ablation electrode and the shaft electrode are configured to form an electrode pair for bipolar ablation.

In Example 26, in the catheter Example 16, at least one of the spline sensing electrodes is configured to be operable as a second ablation electrode.

In Example 27, in the catheter of Example 26, the at least one of the spline sensing electrodes is and the first ablation electrode are configured to operate together as a monopolar ablation electrode.

In Example 28, in the catheter of Example 16, the spline retainer includes a proximal spline retainer mechanically abutted against the support member proximal portions of each of the plurality of splines so as to space apart the spline proximal portions in the expanded and collapsed states.

In Example 29, in the catheter of Example 28, the proximal spline retainer is coupled to and extends from the shaft distal end into an interior space formed by the electrode assembly.

In Example 30, in the catheter of Example 28, the proximal spline retainer is integral to the shaft distal end.

Example 31 is a catheter for ablation of cardiac tissue through irreversible electroporation, the catheter comprising a a tubular shaft having a proximal portion and a distal end; an electrode assembly extending from the distal end of the shaft, the electrode assembly including a plurality of splines each having a spline proximal portion secured to the shaft, and an opposite spline distal portion; an electrical insulating member covering the spline proximal portion, the insulating member having an insulating member proximal portion coupled to the shaft distal end; a plurality of sensing electrodes disposed along the insulating member; and a spline retainer mechanically engaged with at least one of the spline proximal portion and the spline distal portion, the spline retainer adapted to mechanically support the splines.

In Example 32, in the catheter of Example 31, the spline retainer includes a distal spline retainer mechanically coupled to a distal end of each spline distal portion so as to mechanically couple the spline distal portions.

In Example 33, in the catheter of Example 32, the distal spline retainer is electrically conductive and is configured to form a part of the first ablation electrode.

In Example 34, the catheter of Example 33, the distal spline retainer is a conductive ball.

In Example 35, in the catheter of Example 31, the spline retainer is coupled to and extends from the shaft distal end into an interior space formed by the electrode assembly.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a patient, and for treating a heart of the patient, using an electrophysiology system, in accordance with embodiments of the subject matter of the disclosure.

FIG. 2A is a side view of an example electroporation catheter that can be used with example electrophysiology system of FIG. 1.

FIG. 2B is a perspective view of the example catheter of FIG. 2A.

FIG. 3 is a perspective view of a portion of a feature of an example catheter that can be used with example electrophysiology system of FIG. 1.

FIG. 4 is a perspective view of an example electroporation catheter that can be used with example electrophysiology system of FIG. 1 including features that can also be incorporated into the example electroporation catheter of FIGS. 2A-2B.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but yet still cooperate or interact with each other.

Throughout the present disclosure and in the claims, numeric terminology, such as first and second, is used in reference to various components or features. Such use is not intended to denote an ordering of the components or features. Rather, numeric terminology is used to assist the reader in identifying the component or features being referenced and should not be narrowly interpreted as providing a specific order of components or features.

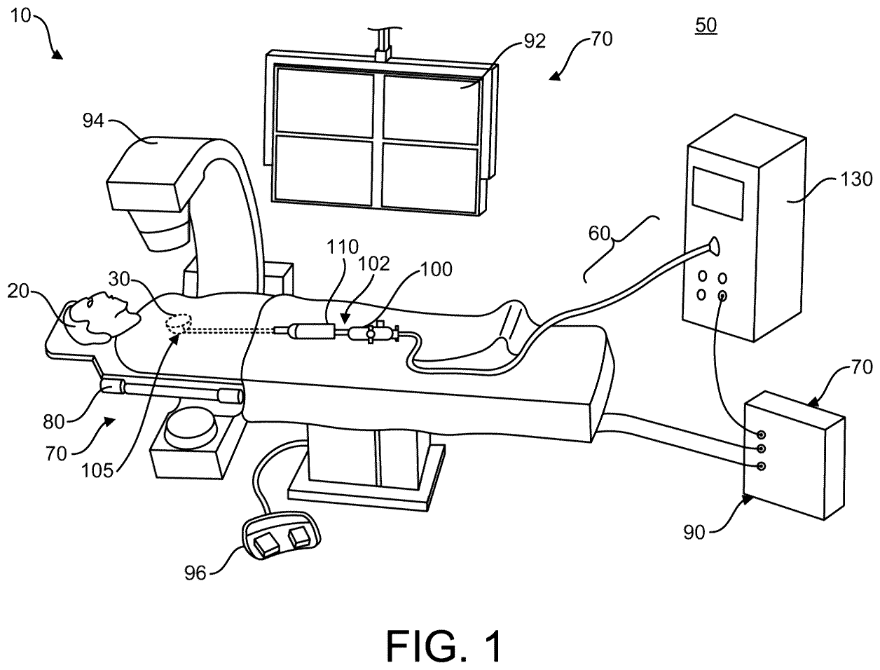

FIG. 1 is a diagram illustrating an exemplary clinical setting 10 for treating a patient 20, and for treating a heart 30 of the patient 20, using an electrophysiology system 50, in accordance with embodiments of the subject matter of the disclosure. The electrophysiology system 50 includes an electroporation catheter system 60 and an electro-anatomical mapping (EAM) system 70, which includes a localization field generator 80, a mapping and navigation controller 90, and a display 92. Also, the clinical setting 10 includes additional equipment such as imaging equipment 94 (represented by the C-arm) and various controller elements, such as a foot controller 96, configured to allow an operator to control various aspects of the electrophysiology system 50. As will be appreciated by the skilled artisan, the clinical setting 10 may have other components and arrangements of components that are not shown in FIG. 1.

The electroporation catheter system 60 includes an electroporation catheter 100 having a proximal portion 102 and a distal portion 105, an introducer sheath 110, and an electroporation console 130. Additionally, the electroporation catheter system 60 includes various connecting elements, e.g., cables, umbilicals, and the like, that operate to functionally connect the components of the electroporation catheter system 60 to one another and to the components of the EAM system 70. This arrangement of connecting elements is not of critical importance to the present disclosure, and the skilled artisan will recognize that the various components described herein can be interconnected in a variety of ways.

In embodiments, the introducer sheath 110 is operable to provide a delivery conduit through which the electroporation catheter 100, in particular all or part of the distal portion 105 thereof, can be deployed to the specific target sites within the patient's heart 30.

In embodiments, the electroporation catheter system 60 is configured to deliver electric field energy to targeted tissue in the patient's heart 30 to create tissue apoptosis, rendering the tissue incapable of conducting electrical signals.

The electroporation console 130 is configured to control functional aspects of the electroporation catheter system 60. In embodiments, the electroporation console 130 includes one or more controllers, microprocessors, and/or computers that execute code out of memory to control and/or perform the functional aspects of the electroporation catheter system 60. In embodiments, the memory can be part of the one or more controllers, microprocessors, and/or computers, and/or part of memory capacity accessible through a network, such as the world wide web. In embodiments, the electroporation console 130 includes pulse generator hardware, software and/or firmware configure to generate electrical pulses in predefined waveforms, which are transmitted to electrodes on the electroporation catheter 100 to generate electric fields sufficient to achieve the desired clinical effect, in particular ablation of target tissue through irreversible electroporation. In embodiments, the electroporation console 130 can deliver the pulsed waveforms to the electroporation catheter 100 in a monopolar or bipolar mode of operation, as will be described in further detail herein.

The EAM system 70 is operable to track the location of the various functional components of the electroporation catheter system 60, and to generate high-fidelity three-dimensional anatomical and electro-anatomical maps of the cardiac chambers of interest. In embodiments, the EAM system 70 can be the RHYTHMIA™ HDx mapping system marketed by Boston Scientific Corporation. Also, in embodiments, the mapping and navigation controller 90 of the EAM system 70 includes one or more controllers, microprocessors, and/or computers that execute code out of memory to control and/or perform functional aspects of the EAM system 70, where the memory, in embodiments, can be part of the one or more controllers, microprocessors, and/or computers, and/or part of memory capacity accessible through a network, such as the world wide web.

As will be appreciated by the skilled artisan, the depiction of the electrophysiology system 50 shown in FIG. 1 is intended to provide a general overview of the various components of the system 50 and is not in any way intended to imply that the disclosure is limited to any set of components or arrangement of the components. For example, the skilled artisan will readily recognize that additional hardware components, e.g., breakout boxes, workstations, and the like, can and likely will be included in the electrophysiology system 50.

The EAM system 70 generates a localization field, via the field generator 80, to define a localization volume about the heart 30, and one or more location sensors or sensing elements on the tracked device(s), e.g., the electroporation catheter 100, generate an output that can be processed by the mapping and navigation controller 90 to track the location of the sensor, and consequently, the corresponding device, within the localization volume. In the illustrated embodiment, the device tracking is accomplished using magnetic tracking techniques, whereby the field generator 80 is a magnetic field generator that generates a magnetic field defining the localization volume, and the location sensors on the tracked devices are magnetic field sensors.

In other embodiments, impedance tracking methodologies may be employed to track the locations of the various devices. In such embodiments, the localization field is an electric field generated, for example, by an external field generator arrangement, e.g., surface electrodes, by intra-body or intra-cardiac devices, e.g., an intracardiac catheter, or both. In these embodiments, the location sensing elements can constitute electrodes on the tracked devices that generate outputs received and processed by the mapping and navigation controller 90 to track the location of the various location sensing electrodes within the localization volume.

In embodiments, the EAM system 70 is equipped for both magnetic and impedance tracking capabilities. In such embodiments, impedance tracking accuracy can, in some instances be enhanced by first creating a map of the electric field induced by the electric field generator within the cardiac chamber of interest using a probe equipped with a magnetic location sensor, as is possible using the aforementioned RHYTHMIA HDx™ mapping system. One exemplary probe is the INTELLAMAP ORION™ mapping catheter marketed by Boston Scientific Corporation.

Regardless of the tracking methodology employed, the EAM system 70 utilizes the location information for the various tracked devices, along with cardiac electrical activity acquired by, for example, the electroporation catheter 100 or another catheter or probe equipped with sensing electrodes, to generate, and display via the display 92, detailed three-dimensional geometric anatomical maps or representations of the cardiac chambers as well as electro-anatomical maps in which cardiac electrical activity of interest is superimposed on the geometric anatomical maps. Furthermore, the EAM system 70 can generate a graphical representation of the various tracked devices within the geometric anatomical map and/or the electro-anatomical map.

Embodiments of the present disclosure provide systems, devices, and methods for selective and rapid application of pulsed electric fields to ablate tissue by irreversible electroporation. Generally, the systems, devices, and methods described herein may be used to generate large electric field magnitudes at desired regions of interest and reduce peak electric field values elsewhere in order to reduce unnecessary tissue damage and electrical arcing. An irreversible electroporation system as described herein may include a signal generator and a processor configured to apply one or more voltage pulse waveforms to a selected set of electrodes of an ablation device to deliver energy to a region of interest (e.g., ablation energy for a set of tissue in a pulmonary vein ostium or antrum). The pulse waveforms disclosed herein may aid in therapeutic treatment of a variety of cardiac arrhythmias (e.g., atrial fibrillation). In order to deliver the pulse waveforms generated by the signal generator, one or more electrodes of the ablation device may have an insulated electrical lead configured for sustaining a voltage potential in the order of several hundred volts to several thousand volts. The electrodes may be independently addressable such that each electrode may be controlled (e.g., deliver energy) independently of any other electrode of the device. In this manner, the electrodes may deliver different energy waveforms with different timing synergistically for electroporation of tissue.

Pulse waveforms for electroporation energy delivery as disclosed herein may enhance the safety, efficiency and effectiveness of energy delivery to tissue by reducing the electric field threshold associated with irreversible electroporation, thus yielding more effective ablative lesions with a reduction in total energy delivered. In some embodiments, the voltage pulse waveforms disclosed herein may be hierarchical and have a nested structure. For example, the pulse waveform may include hierarchical groupings of pulses having associated timescales. In some embodiments, the methods, systems, and devices disclosed herein may comprise one or more of the methods, systems, and devices described in International Application Serial No. PCT/US2016/057664, filed on Oct. 19, 2016, and titled “SYSTEMS, APPARATUSES AND METHODS FOR DELIVERY OF ABLATIVE ENERGY TO TISSUE,” the contents of which are hereby incorporated by reference in its entirety.

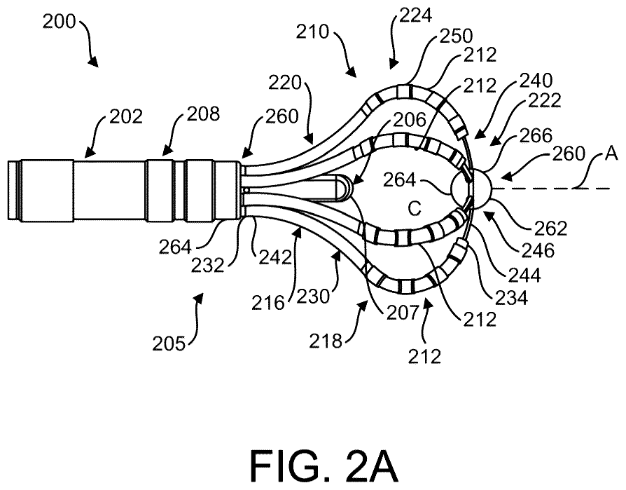

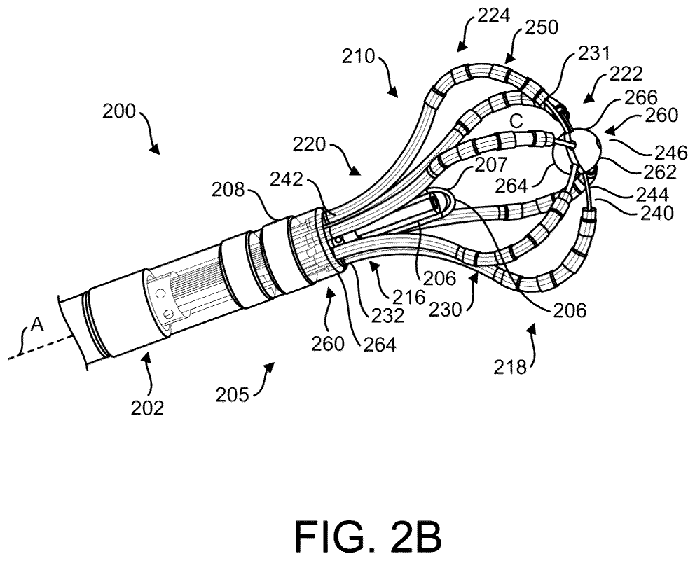

FIGS. 2A and 2B illustrate multiple views of an embodiment of an electroporation catheter 200, or the distal region 205 of the electroporation catheter 200, for ablation of cardiac tissue, which can be an example of catheter 100 and used with the electrophysiology system 50. The electroporation catheter 200 includes a tubular shaft 202 defining a longitudinal axis A and having a distal end 204 of the shaft 202 and an opposite, proximal end of the shaft. The longitudinal axis A is presented as a line passing through a centroid of a cross section of the shaft 202. A distal region 205 of the catheter 200 is configured to be deployed proximate target tissue, such as within a chamber of the patient's heart. An expandable electrode assembly 210 is coupled to the shaft distal end 204 and configured to generate an electric field. In one example, the electric field effects an irreversible electroporation in selected cardiac tissue. The electrode assembly 210 is transitionable between the collapsed state and the expanded state. In the expanded state, the electrode assembly 210 in the expanded state includes a maximum radial dimension greater than a maximum radial dimension of the distal end 204 of the elongated shaft 202.

The electrode assembly 210 includes a plurality of longitudinally extending splines 212 formed into a spherical basket 214 in the expanded state. Spherical, as used in this disclosure to define the shape of the electrode assembly in the expanded state, includes spheres, spheroids (as an approximate spherical body), ellipsoids, and other three-dimensional analogues of curvilinear shapes with or without circular symmetry. Each of the splines 212 includes a proximal portion 220 secured to the shaft 202 and an opposite distal portion 222, and a central portion 224 therebetween. Each of the splines 212 includes an electrical insulating member 230 having an insulating member proximal end 232 secured to the shaft distal end 204 and an insulating member distal end 234 opposite the insulating member proximal end 232.

Each of the splines 212 further includes an electrically conductive support member 240 partially disposed with the insulating member 230 having a support member proximal end 242 disposed within the insulating member 230 and a support member distal portion 244 extending distally from the insulating member 230 and terminating at a support member distal end 246 or distal tip. Each of the conductive support members 240 is configured to be electrically coupled to the external electroporation console 130 including a pulse generator so as to be operable as a first ablation electrode configured to generate the electric field when a pulsed waveform is delivered to each support member 240. The electrode assembly 210 further includes a plurality of spline sensing electrodes 250 disposed along the insulating member 230.

The catheter 200 includes a spline retainer 260 that is mechanically engaged with at least one of the proximal portions 220 or the distal portions 222 of the splines 212. In the illustrated example, the spline retainer 260 includes a distal spline retainer 262 that is mechanically coupled to the conductive support member distal end 246 of each of the splines 212 so as to mechanically couple together the spline distal portions 222. Further, the spline retainer 260 includes a proximal spline retainer 264 that is mechanically abutted against the spline proximal portions 220 of each of the splines 212 so as to space apart the spline proximal portions 220 in the expanded and collapsed states.

In various embodiments, the elongate shaft 202 is formed of a biocompatible material that provide sufficient sturdiness and flexibility to allow the shaft 202 to be navigated through the vasculature of a patient and reach the treatment site, such as a chamber of the heart. In some embodiments, the shaft 202 is formed of multiple different materials to provide the electroporation catheter 200 with more flexibility at the distal end 204 than a proximal end. Further, the shaft 202 can included a tubular woven member to provide torsional stiffness and bending flexibility. The shaft 202 can include various markers for use with a visualization system, such as radiopaque or echogenic markers, or EAM electrodes to facilitate visualization.

The catheter shaft 202 can also accommodate pull wires to deflect the electrode assembly 210 to the treatment site. The distal end 204 can include sensors such as tracking sensors and force sensors and additional elements such as an irrigation element. In the particular illustrated embodiment, the electroporation catheter 200 includes a central post 206 extending distally from the distal end 204 of the shaft 202. As shown, the central post 206 extends partially into an inner space of the electrode assembly 210 and includes a post electrode 207. An optional irrigation lumen (not shown) is supported by the central post 206. In embodiments, the central post 206 may house additional components. For example, in embodiments, a magnetic navigation sensor (not shown) may be partially or wholly disposed within the central post 206. However, in other embodiments such a sensor may be located elsewhere on the electroporation catheter 200 (e.g., within the shaft 202). The post electrode 207 can provide a number of functional advantages. In one example, the post electrode 207 can operate as a reference for unipolar electrograms, in lieu of reliance on surface ECG patch electrodes as are otherwise known in the art. The location of the post electrode 207 for this purpose positions the reference electrode much closer to the tissue being sensed than is possible with the conventional surface ECG approach, which may advantageously minimize far field noise and provide much sharper unipolar electrograms than what are possible using surface ECG electrodes. The post electrode 207 may also be operable to sense and measure other electrical parameters, e.g., voltages between it and the ablation electrodes or other sensing electrodes on the electrode assembly 210, thereby providing data usable for, in some examples, determining the shape of the electrode assembly during use (including when deformed by forces applied by cardiac walls), and displaying shape information via the EAM system 70.

In some embodiments, the distal end 204 of the shaft 202 includes an exposed shaft electrode 208, such as plurality of exposed shaft electrodes, proximate the electrode assembly 210. The exposed shaft electrode 208 in embodiments can be used with the electrode assembly 210 and configured as a return electrode in a bipolar mode.

The electrode assembly 210 in some embodiments includes a coupling portion 216 and a deformable portion 218 of the basket. The coupling portion 216 of the electrode assembly 210 is secured to the distal end 204 of the shaft 202, and the deformable portion 218 extends distally from the coupling portion 216. The deformable portion 218 of the electrode assembly 210 can be collapsed for delivery, such as through an introducer sheath, and expanded for treatment at the treatment site. The coupling portion 216 is mechanically coupled, either directly or indirectly, to the catheter shaft 202. The coupling portion 216 can include components to directly coupled to the shaft 202 or coupled to a transitional part coupled to the shaft 202. The coupling portion 216 of the electrode assembly 210 is electrically coupled to the conductive paths, such as wires, extending along the shaft 202 to the shaft proximal end.

In use with electrophysiology system 50, the electroporation console 130 is electrically coupled via the conductive paths in the shaft 202 to the coupling portion 216 of the electrode assembly 210. The deformable portion 218 of the electrode assembly 210 includes the proximal portion 220 mechanically and electrically coupled to the coupling portion 216. In various embodiments, the deformable portion 218 defines open area through which blood or other fluids can flow. For example, the splines 212 form a concave shape around an internal cavity C, and the configuration of the basket provides for openings between the splines 212. The deformable portion 218 of the electrode assembly includes a cross-sectional dimension, such as a dimension taken generally perpendicular to the axis A, that is larger than a cross section dimension of the shaft 202. The electrode assembly 210 can provide for wider lesions via electroporation to the tissue in a shorter period of time to create a pattern of overlapping lesions on the tissue reducing the likelihood of arrhythmogenic gaps, or the tip can facilitate the delivery of more power to provide for deeper lesions than catheter shaft electrodes.

The insulator 230 is an electrical insulator that resists delivery of electrical energy on the electrode assembly 210 through the insulator 230. The insulator 230 is disposed on at least the proximal portion 220 of the splines 212 and surrounds the conductive support members 240. For example, the insulator 230 extends completely around the support members 240 proximal to the distal portions 244 in the embodiment and does not provide radial gaps of insulation to expose the support members 240. The insulator 230 continuously extends from the proximal end 222 to the central portion 224 of the splines 212. For example, the insulator 230 does not include longitudinal gaps to expose the conductive support members 240 between the insulating member proximal end 232 and the insulating member distal end 234. In some embodiments, the insulator 230 is constructed from a polymer applied to the conductive support members 240 such as via a dip coating or spray coating the spline 212. In some embodiments, the insulator 230 is tubular polymer such as a shrink fit polymer disposed about the conductive support members 240 and attached in place. In addition to the proximal portion 220, the insulator can be extended distally to the central portion 224, such as distal to the maximum radial dimension of the hull 212.

The conductive support members 240 provide a conductive structure that can be configured with system 50 as a single ablation electrode. The conductive support members 240 receive electrical energy and is configured to generate the electric field to effect an irreversible electroporation in selected cardiac tissue. In embodiments, the conductive support members 240 are axial elements that include thin conductors to form the structure of the splines 212 and define the open area. Examples of conductive support members 240 include flexible round wires, pins, straps of metal, strips, flat wires, and planks. In some embodiments, the conductive support members 240 can be formed from a conductive shape memory material such as a nickel titanium alloy that will transition to the expanded state once a constrictor device applying an axial force is removed or from another conductive material that is expanded and contracted via a controllable mechanism.

In some embodiments, the electrode assembly 210 includes one or more additional electrodes, such as sensing or mapping electrodes 250 in addition to the conductive support members 240. For example, a plurality of sensing electrodes 250 can be attached to the insulating members 230 at selected locations and electrically isolated from the conductive support members 240. In the illustrated embodiments, the sensing electrodes 250 are be spaced apart from each other each spine 212. The sensing electrodes 250 are configured for, among other things, sensing cardiac electrical signals, localization of the electrode assembly 210 within the patient anatomy such as via the EAM system 70, and to determine proximity to target tissue within the anatomy. The sensing electrodes 250 in the electrode assembly 210 can be electrically coupled to a one or more lead conductors that extends the length of the shaft 202 that are configured to carry an electrical signal received at the sensing electrode 250. In an embodiment, one or more sensing electrodes 250 can be configured to carry the electrical signal provided to the conductive support members 240 and operate together as ablation electrodes such as in a monopolar mode. In another embodiment, one or more sensing electrodes 250 can be configured to carry another electrical signal provided to the at least one sensing electrode 250 and operate as a second ablation electrode.

In the illustrated example, the spline retainer 260 includes a distal spline retainer 262 and a proximal spline retainer 264. Embodiments of the catheter 100 can include a distal spline retainer, a proximal spline retainer, or both. The distal spline retainer 262 is mechanically coupled to the conductive support member distal end 246 of each of the splines 212 so as to mechanically couple together the spline distal portions 222. In embodiments, the distal spline retainer 262 is electrically conductive and is electrically coupled to one or more of the support members 240 and configured to form a part of the first ablation electrode. In some embodiments, the distal spline retainer 262 is configured to be operable as a distal sensing electrode and is electrically isolated from the conductive members 240. The distal spline retainer 262 is disposed at a distal end 246 of the electrode assembly, and in the illustrated embodiment is a conductive ball. In one embodiment, the distal spline retainer 262 is a multipiece assembly having a first piece 264 proximal to the distal end 246 mechanically coupled to a second piece 266 distal to the distal end 246. The distal spline retainer 262 can include a plurality of openings 268 disposed around a circumference of the assembly to receive the conductive support members 240. The proximal spline retainer 264 that is mechanically abutted against the spline proximal portions 220 of each of the splines 212 so as to space apart the spline proximal portions 220 in the expanded and collapsed states. In the illustrated example, the proximal spline retainer 264 can include a cap near the distal portion 204 of the shaft 202 with a plurality of openings to receive splines 212 into the shaft 202.

In various embodiments, the catheter 200 is configured to be operated in a bipolar mode to generate an electric field for electroporation. During bipolar operation of the catheter 200, the splines 212 is configured as the anode (or cathode) and a second set of one or more electrodes at the distal portion 205 of the catheter 200, such as exposed shaft electrode 208, is configured as the cathode (or anode), to generate an electrical field. In this example, a pad dispersive electrode described above is not used, and the electrical field is not typically extended through the patient's body, but rather through a localized portion of tissue proximate the electrode assembly 210. In embodiments, the catheter 200 is also configured to be operated in a monopolar mode to generate an electric field for electroporation. For example, the splines 212 can be electrically coupled to a single lead conductor that extends the length of the shaft 202, or to a set of lead conductors in the shaft 202 that are configured to carry the same electrical signal to generate the electric field. The splines 212 is configured as one of the anode or the cathode. None of the electrodes in the electrode assembly 210 or the distal end 204 of the catheter shaft 202 are configured as the other of the cathode or the anode. Instead, the other of the cathode or the anode is provided in the form of a pad dispersive electrode located on the patient during electroporation. The electrical field is formed between the exposed conductive portions of the splines and any other activated electrodes of the electrode assembly 210 or distal end 204 and the pad dispersive electrode.

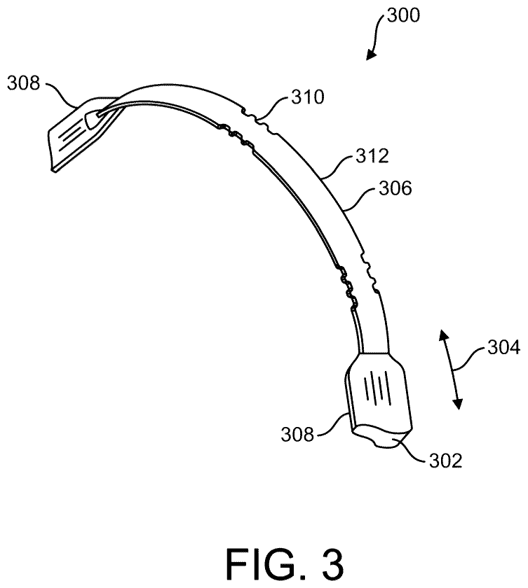

FIG. 3 illustrates a section of a conductive support member 300 that can be incorporated into catheter 100, such as in the electrode assembly 210 as conductive support member 240. The conductive support members 300 can include features to help reduce strain on the splines 212 in the expanded and collapsed states and during transitions between the expanded and collapsed states. In certain embodiments, the conductive support members 300 can have a plurality of cross-sectional geometries 302 extending along the length 304, such as thinner or flatter longitudinal sections 306 in otherwise round cross sections 308 that help reduce strain. In some embodiments, the conductive support members 300 can have varied (two-dimensional) profiles extending along the length 304, such as side cutouts 310 in otherwise straight sides 312. Various combinations of none or more cross-sectional geometries or profiles can be included in one or more conductive support members.

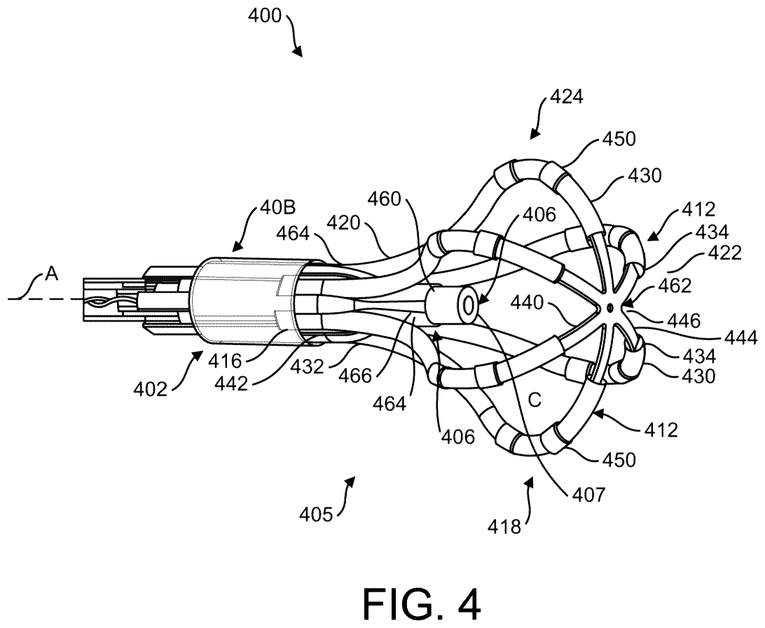

FIG. 4 illustrates an embodiment of an electroporation catheter 400, or the distal region 405 of the electroporation catheter 400, for ablation of cardiac tissue, which can be an example of catheter 100 and used with the electrophysiology system 50. Electroporation catheter 400 can include features and advantages of electroporation catheter 200. The electroporation catheter 400 includes a tubular shaft 402 defining a longitudinal axis A and having a distal end 404 of the shaft 402 and an opposite, proximal end of the shaft. The longitudinal axis A is presented as a line passing through a centroid of a cross section of the shaft 402. A distal region 405 of the catheter 400 is configured to be deployed proximate target tissue, such as within a chamber of the patient's heart. An expandable electrode assembly 410 is coupled to the shaft distal end 404 and configured to generate an electric field. In one example, the electric field effects an irreversible electroporation in selected cardiac tissue.

The electrode assembly 410 is transitionable between the collapsed state and the expanded state. In the expanded state, the electrode assembly 410 in the expanded state includes a maximum radial dimension greater than a maximum radial dimension of the distal end 404 of the elongated shaft 402. The electrode assembly 410 includes a plurality of longitudinally extending splines 412 formed into a spherical basket 414 in the expanded state. Each of the splines 412 includes a proximal portion 420 secured to the shaft 402 and an opposite distal portion 422, and a central portion 424 therebetween.

Each of the splines 412 includes an electrical insulating member 430 having an insulating member proximal end 432 secured to the shaft distal end 404 and an insulating member distal end 434 opposite the insulating member proximal end 432. Each of the splines 412 further includes an electrically conductive support member 440 partially disposed with the insulating member 430 having a support member proximal end 442 disposed within the insulating member 430 and a support member distal portion 444 extending distally from the insulating member 430 and terminating at a support member distal end 446. Each of the conductive support members 440 is configured to be electrically coupled to the external electroporation console 130 including a pulse generator so as to be operable as a first ablation electrode configured to generate the electric field when a pulsed waveform is delivered to each support member 440. The electrode assembly 410 further includes a plurality of spline sensing electrodes 450 disposed along the insulating member 430. Features of the insulating member 430 are described above with reference to insulating member 230.

The catheter 400 includes a spline retainer 460 that is mechanically engaged with at least one of the proximal portions 420 or the distal portions 422 of the splines 412. In the illustrated example, the spline retainer 460 includes a distal spline retainer 462 and a proximal spline retainer 464. The proximal spline retainer 464 in the example is coupled to a shaft distal end 404 and is mechanically abutted against the proximal portions 420 of each of the splines 412 so as to space apart the spline proximal portions 420 in the expanded and collapsed states.

The catheter shaft distal end 404 can include sensors such as tracking sensors and force sensors and additional elements such as an irrigation element. In the particular illustrated embodiment, the electroporation catheter 400 includes a central post 406 extending distally from the distal end 404. As shown, the central post 406 extends partially into an inner space of the electrode assembly 410 and includes a post electrode 407. An optional irrigation lumen (not shown) is supported by the central post 206. In embodiments, the central post 406 may house additional components. For example, in embodiments, a magnetic navigation sensor (not shown) may be partially or wholly disposed within the central post 406. However, in other embodiments such a sensor may be located elsewhere on the electroporation catheter 400 (e.g., within the shaft 402). As described with reference to the post electrode 207 above, the post electrode 407 can operate as a reference for unipolar electrograms, in lieu of reliance on surface ECG patch electrodes as are otherwise known in the art and may also be operable to sense and measure other electrical parameters, e.g., voltages between it and the ablation electrodes or other sensing electrodes on the electrode assembly 410, thereby providing data usable for, in some examples, determining the shape of the electrode assembly during use (including when deformed by forces applied by cardiac walls), and displaying shape information via the EAM system 70.

In some embodiments, the distal end 404 of the shaft 402 includes an exposed shaft electrode 408, such as plurality of exposed shaft electrodes, proximate the electrode assembly 410. The exposed shaft electrode 408 in embodiments can be used with the electrode assembly 410 and configured as a return electrode in a bipolar mode.

The electrode assembly 410 in embodiments includes a coupling portion 416 and a deformable portion 418 of the basket. The coupling portion 416 of the electrode assembly 410 is secured to the distal end 404 of the shaft 402, and the deformable portion 418 of the electrode assembly 410 extends distally from the coupling portion 416. The deformable portion 418 of the electrode assembly 410 can be collapsed for delivery, such as through an introducer sheath, and expanded for treatment at the treatment site. The coupling portion 416 of the electrode assembly 410 is mechanically coupled, either directly or indirectly, to the catheter shaft 402. The coupling portion 416 is electrically coupled to the conductive paths, such as wires, extending along the shaft 402 to the shaft proximal end. In use with electrophysiology system 50, the electroporation console 130 is electrically coupled via the conductive paths in the shaft 402 to the coupling portion 416. The deformable portion 418 of the electrode assembly 410 includes the proximal portion 420 mechanically and electrically coupled to the coupling portion 416. The deformable portion 418 of the electrode assembly 410 includes a cross-sectional dimension, such as a dimension taken generally perpendicular to the axis A, that is larger than a cross section dimension of the shaft 402.

In the illustrated example, the spline retainer 460 includes a distal spline retainer 462 and a proximal spline retainer 464. Embodiments of the catheter 100 can include a distal spline retainer, a proximal spline retainer, or both. The distal spline retainer 462 is mechanically coupled to the conductive support member distal end 246 of each of the splines 212 so as to mechanically couple together the spline distal portions 222. In embodiments, the distal spline retainer 262 is electrically conductive and is electrically coupled to one or more of the support members 240 and configured to form a part of the first ablation electrode. In some embodiments, the distal spline retainer 262 is configured to be operable as a distal sensing electrode and is electrically isolated from the conductive members 240. The distal spline retainer 262 is disposed at a distal end 246 of the electrode assembly, and in the illustrated embodiment is a conductive hub. In the illustrated example, the proximal spline retainer 464 is mechanically abutted against the each of the splines 412 so as to space apart the spline proximal portions 420 in the expanded and collapsed states. In the illustrated example, the proximal spline retainer 464 is configured to abut against a proximal section of the deformable portion 418 of the electrode assembly 410. For example, the central post 406 is configured to include a plurality of longitudinally extending channels, grooves or detents 466 to receive at least an inner surface of the splines 412 such as when the electrode assembly is in the collapsed state and to allow at least portions of the splines 412 to spread apart from grooves 466 when in the expanded state. The grooves 466 are configured to engage the deformable portion 418 of the electrode assembly 410 in at least the collapsed configuration to space apart the spline proximal portions 420 from each other and relieve strain. In the illustrated embodiment, the grooves 466 also at least partially engage the deformable portion 418 of the electrode assembly 410 in the expanded state.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is to be construed under the provisions of 35 U.S.C. 112 (f), unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A catheter for ablation of cardiac tissue through irreversible electroporation, the catheter comprising:
a tubular shaft having a proximal portion and a distal end;
an electrode assembly extending from the distal end of the shaft, the electrode assembly including a plurality of splines each having a spline proximal portion secured to the shaft, and an opposite spline distal portion, each of the splines comprising:
an electrically conductive support member having a support member proximal portion coupled to the shaft distal end and a support member distal portion terminating at a support member distal end, wherein each of the electrically conductive support members is configured to be operable as a first ablation electrode configured to generate an electric field when a pulsed waveform is delivered to each support member; and
an electrical insulating member covering the support member proximal portion, the insulating member having an insulating member proximal portion coupled to the shaft distal end; and
a plurality of sensing electrodes disposed along the insulating member; and
a spline retainer mechanically engaged with at least one of the support member distal portion or the support member proximal portion of each of the plurality of splines, the spline retainer adapted to mechanically support the splines.

2. The catheter of claim 1, wherein the spline retainer includes a distal spline retainer mechanically coupled to the support member distal end of each of the plurality of splines so as to mechanically couple the spline distal portions together.

3. The catheter of claim 2, wherein the distal spline retainer is electrically conductive and is electrically coupled to one or more of the support members and configured to form a part of the first ablation electrode.

4. The catheter of claim 2, wherein the distal spline retainer is configured to be operable as a distal sensing electrode.

5. The catheter of claim 4, wherein the distal spline retainer is disposed at a distal tip of the electrode assembly.

6. The catheter of claim 5, wherein the distal spline retainer is a conductive ball.

7. The catheter of claim 5, wherein the distal spline retainer includes a first piece proximal to the distal tip mechanically coupled to a second piece distal to the distal tip.

8. The catheter of claim 1, wherein the first ablation electrode is configured as a monopolar ablation electrode.

9. The catheter of claim 1, further comprising a shaft electrode disposed on the shaft proximate the distal end thereof.

10. The catheter of claim 9, wherein the first ablation electrode and the shaft electrode are configured to form an electrode pair for bipolar ablation.

11. The catheter of claim 1, wherein at least one of the spline sensing electrodes is configured to be operable as a second ablation electrode.

12. The catheter of claim 11, wherein the at least one of the spline sensing electrodes is and the first ablation electrode are configured to operate together as a monopolar ablation electrode.

13. The catheter of claim 1, wherein the spline retainer includes a proximal spline retainer mechanically abutted against the support member proximal portions of each of the plurality of splines so as to space apart the spline proximal portions in the expanded and collapsed states.

14. The catheter of claim 13, wherein the proximal spline retainer is coupled to and extends from the shaft distal end into an interior space formed by the electrode assembly.

15. The catheter of claim 13, wherein the the proximal spline retainer is integral to the shaft distal end.

16. A catheter for ablation of cardiac tissue through irreversible electroporation, the catheter comprising:
a tubular shaft having a proximal portion and a distal end;
an electrode assembly extending from the distal end of the shaft, the electrode assembly including a plurality of splines each having a spline proximal portion secured to the shaft, and an opposite spline distal portion;
an electrical insulating member covering the spline proximal portion, the insulating member having an insulating member proximal portion coupled to the shaft distal end;
a plurality of sensing electrodes disposed along the insulating member; and
a spline retainer mechanically engaged with at least one of the spline proximal portion and the spline distal portion, the spline retainer adapted to mechanically support the splines.

17. The catheter of claim 16, wherein the spline retainer includes a distal spline retainer mechanically coupled to a distal end of each spline distal portion so as to mechanically couple the spline distal portions.

18. The catheter of claim 17, wherein the distal spline retainer is electrically conductive and is configured to form a part of the first ablation electrode.

19. The catheter of claim 18, wherein the distal spline retainer is a conductive ball.

20. The catheter of claim 16, wherein the spline retainer is coupled to and extends from the shaft distal end into an interior space formed by the electrode assembly.
